(19) United States
(12) Patent Application Publication
(10) Pub. No.: US 2018/0330496 A1
(43) Pub. Date: Nov. 15, 2018

(54) Generation Of Personalized Surface Data

(71) Applicant: Siemens Healthcare GmbH, Erlangen (DE)

(72) Inventors: Kai Ma, West Windsor, NJ (US); Vivek Kumar Singh, Princeton, NJ (US); Birgi Tamersoy, Erlangen (DE); Yao-jen Chang, Princeton, NJ (US); Terrence Chen, Princeton, NJ (US)

(21) Appl. No.: 15/592,403

(22) Filed: May 11, 2017

(51) Int. Cl. G06T 7/00; H04N 13/02; H04N 13/00; G06T 17/20; A61B 6/03; A61B 6/00; A61B 34/10

(52) U.S. Cl. CPC G06T 7/0012; H04N 13/0239; H04N 13/004; A61B 2034/108; A61B 6/03; A61B 6/52; A61B 34/10; G06T 17/205

(57) ABSTRACT

A system and method includes acquisition of first surface data of a patient in a first pose using a first imaging modality, acquisition of second surface data of the patient in a second pose using a second imaging modality, combination of the first surface data and the second surface data to generate combined surface data, for each point of the combined surface data, determination of a weight associated with the first surface data and a weight associated with the second surface data, detection of a plurality of anatomical landmarks based on the first surface data, initialization of a first polygon mesh by aligning a template polygon mesh to the combined surface data based on the detected anatomical landmarks, deformation of the first polygon mesh based on the combined surface data, a trained parametric deformable model, and the determined weights, and storage of the deformed first polygon mesh.

### BACKGROUND

Modeling of human body surfaces is desirable for many technological applications. For example, an efficient and accurate modeling technique may allow estimation of body dimensions and/or body pose based on data representing only a subset of actual body surface. Accordingly, modeling may facilitate the positioning of a patient's body for medical treatment. Some models may facilitate accurate segmentation of an estimated body surface, which may further improve positioning of patient anatomy with respect to treatment devices.

Some surface modeling techniques use statistical models to simulate the shape and pose deformations of a human body surface. A statistical model is initially trained with many three-dimensional datasets, each of which represents a human body surface. These datasets are typically captured using lasers to perceive surface details of an upright human body. The statistical models may be adapted to account for skin surfaces under clothing and skin surface deformation caused by body motions, but improved and/or better-trained models are desired.

### BRIEF DESCRIPTION OF THE DRAWINGS

FIG. 1 illustrates a system according to some embodiments;

FIG. 2 is a flow diagram of process to generate a graph according to some embodiments;

FIG. 3 illustrates surface data according to some embodiments;

FIG. 4 is a block diagram illustrating a process according to some embodiments;

FIG. 5 illustrates surface data according to some embodiments;

FIG. 6 is a flow diagram of process to generate a graph according to some embodiments; and

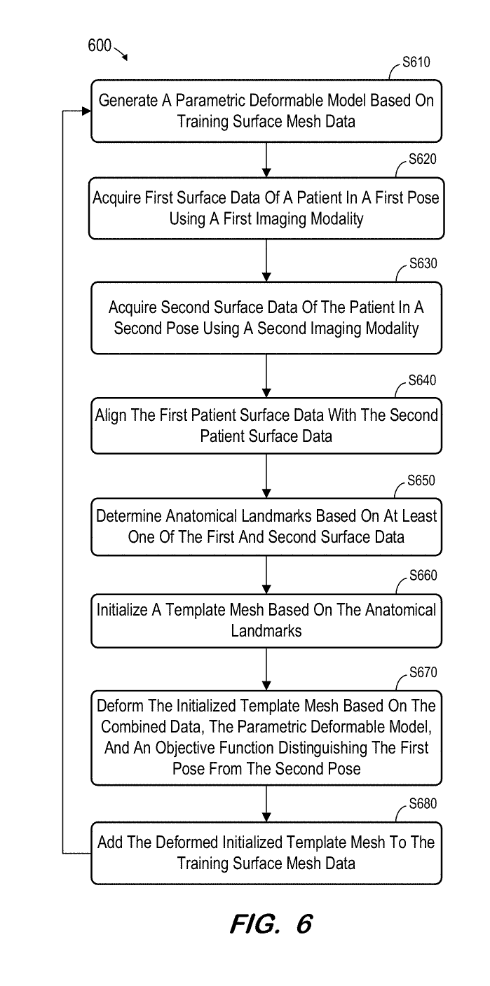

FIG. 7 is a block diagram illustrating a process according to some embodiments.

### DETAILED DESCRIPTION

The following description is provided to enable any person in the art to make and use the described embodiments and sets forth the best mode contemplated for carrying out the described embodiments. Various modifications, however, will remain apparent to those in the art.

Some embodiments provide improved surface data modeling by generating model training data based on surface data obtained from two or more imaging sources. The imaging sources may acquire image data using different imaging modalities (e.g., color sensing, range sensing, thermal sensing, magnetic resonance-based sensing, computed tomography, X-ray, ultrasound). Some embodiments may thereby provide surface data modeling which accounts for soft tissue deformations caused by physical interactions, such as those resulting from lying down on a flat surface.

Some embodiments may be used to generate a personalized three-dimensional mesh of a patient prior to further imaging or treatment. The mesh may be used in place of other images (e.g., topogram, CT images) to predict the position of organs in the patient's body to determine a scan/treatment region. Improved prediction may improve treatment and decrease the exposure of healthy tissue to unnecessary radiation.

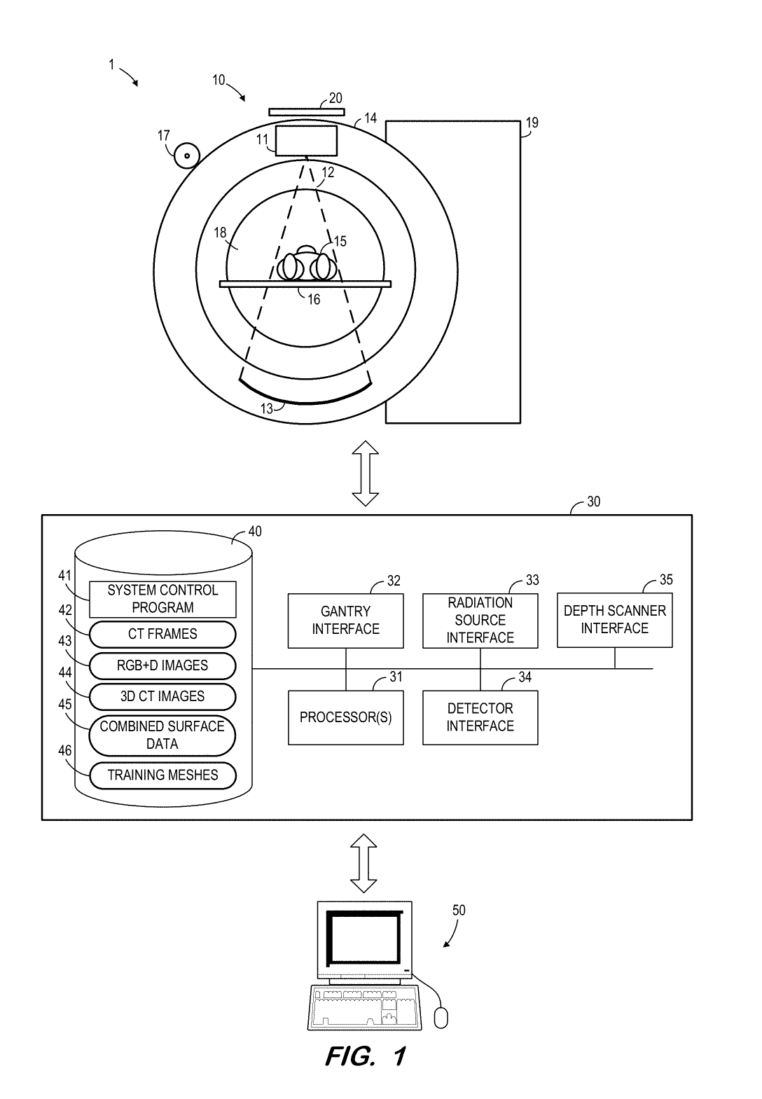

FIG. 1 illustrates system 1 according to some embodiments. System 1 includes x-ray imaging system 10, scanner 20, control and processing system 30, and operator terminal 50. Generally, and according to some embodiments, X-ray imaging system 10 acquires two-dimensional X-ray images of a patient volume and scanner 20 acquires surface images of a patient. Control and processing system 30 controls X-ray imaging system 10 and scanner 20, and receives the acquired images therefrom. Control and processing system 30 processes the images to generate a mesh image as described below. Such processing may be based on user input received by terminal 50 and provided to control and processing system 30 by terminal 50.

Imaging system 10 comprises a CT scanner including X-ray source 11 for emitting X-ray beam 12 toward opposing radiation detector 13. Embodiments are not limited to CT data or to CT scanners. X-ray source 11 and radiation detector 13 are mounted on gantry 14 such that they may be rotated about a center of rotation of gantry 14 while maintaining the same physical relationship therebetween.

Radiation source 11 may comprise any suitable radiation source, including but not limited to a Gigalix™ x-ray tube. In some embodiments, radiation source 11 emits electron, photon or other type of radiation having energies ranging from 50 to 150 keV.

Radiation detector 13 may comprise any system to acquire an image based on received x-ray radiation. In some embodiments, radiation detector 13 is a flat-panel imaging device using a scintillator layer and solid-state amorphous silicon photodiodes deployed in a two-dimensional array. The scintillator layer receives photons and generates light in proportion to the intensity of the received photons. The array of photodiodes receives the light and records the intensity of received light as stored electrical charge.

In other embodiments, radiation detector 13 converts received photons to electrical charge without requiring a scintillator layer. The photons are absorbed directly by an array of amorphous selenium photoconductors. The photoconductors convert the photons directly to stored electrical charge. Radiation detector 13 may comprise a CCD or tube-based camera, including a light-proof housing within which are disposed a scintillator, a mirror, and a camera.

The charge developed and stored by radiation detector 13 represents radiation intensities at each location of a radiation field produced by x-rays emitted from radiation source 11. The radiation intensity at a particular location of the radiation field represents the attenuative properties of mass (e.g., body tissues) lying along a divergent line between radiation source 11 and the particular location of the radiation field. The set of radiation intensities acquired by radiation detector 13 may therefore represent a two-dimensional projection image of this mass.

To generate X-ray images, patient 15 is positioned on bed 16 to place a portion of patient 15 between X-ray source 11 and radiation detector 13. Next, X-ray source 11 and radiation detector 13 are moved to various projection angles with respect to patient 15 by using rotation drive 17 to rotate gantry 14 around cavity 18 in which patient 15 is positioned. At each projection angle, X-ray source 11 is powered by high-voltage generator 19 to transmit X-ray radiation 12 toward detector 13. Detector 13 receives the radiation and produces a set of data (i.e., a raw X-ray image) for each projection angle.

Scanner 20 may comprise a depth camera. The image data obtained from a depth camera may be referred to as RGB-D (RGB+Depth) data, and includes an RGB image, in which each pixel has an RGB (i.e., Red, Green and Blue) value, and a depth image, in which the value of each pixel corresponds to a depth or distance of the pixel from scanner 20. A depth camera may comprise a structured light-based camera (e.g., Microsoft Kinect or ASUS Xtion), a stereo camera, or a time-of-flight camera (e.g., Creative TOF camera) according to some embodiments.

System 30 may comprise any general-purpose or dedicated computing system. Accordingly, system 30 includes one or more processors 31 configured to execute processor-executable program code to cause system 30 to operate as described herein, and storage device 40 for storing the program code. Storage device 40 may comprise one or more fixed disks, solid-state random access memory, and/or removable media (e.g., a thumb drive) mounted in a corresponding interface (e.g., a USB port).

Storage device 40 stores program code of system control program 41. One or more processors 31 may execute system control program 41 to move gantry 14, to move table 16, to cause radiation source 11 to emit radiation, to control detector 13 to acquire an image, to control scanner 20 to acquire an image, and to perform any other function. In this regard, system 30 includes gantry interface 32, radiation source interface 33 and depth scanner interface 35 for communication with corresponding units of system 10.

Two-dimensional X-ray data acquired from system 10 may be stored in data storage device 40 as CT frames 42, in DICOM or another data format. Each frame 42 may be further associated with details of its acquisition, including but not limited to time of acquisition, imaging plane position and angle, imaging position, radiation source-to-detector distance, patient anatomy imaged, patient position, contrast medium bolus injection profile, x-ray tube voltage, image resolution and radiation dosage. Device 40 also stores RGB+D images 43 acquired by scanner 20. An RGB+D image 43 may be associated with a set of CT frames 42, in that the associated image/frames were acquired at similar times while patient 15 was lying in substantially the same position. As will be described below, in some embodiments, an RGB+D image 43 may be associated with a set of CT frames 42 in that both represent a same patient, but disposed in different poses.

Processor(s) 31 may execute system control program 41 to reconstruct three-dimensional CT images 44 from corresponding sets of two-dimensional CT frames 42 as is known in the art. As will be described below, surface data may be determined from such three-dimensional CT images 44 and aligned with a corresponding RGB+D image 43.

Combined surface data 45 may comprise aligned or fused surface CT data and RGB+D data. Training meshes 46 may comprise surface data acquired in any manner that is or becomes known as well as personalized surface data generated according to some embodiments.

Terminal 50 may comprise a display device and an input device coupled to system 30. Terminal 50 may display any of CT frames 42, RGB+D images 43, 3D CT images 44, combined surface data 45 and training meshes 46 received from system 30, and may receive user input for controlling display of the images, operation of imaging system 10, and/or the processing described herein. In some embodiments, terminal 50 is a separate computing device such as, but not limited to, a desktop computer, a laptop computer, a tablet computer, and a smartphone.

Each of system 10, scanner 20, system 30 and terminal 40 may include other elements which are necessary for the operation thereof, as well as additional elements for providing functions other than those described herein.

According to the illustrated embodiment, system 30 controls the elements of system 10. System 30 also processes images received from system 10. Moreover, system 30 receives input from terminal 50 and provides images to terminal 50. Embodiments are not limited to a single system performing each of these functions. For example, system 10 may be controlled by a dedicated control system, with the acquired frames and images being provided to a separate image processing system over a computer network or via a physical storage medium (e.g., a DVD).

Embodiments are not limited to a CT scanner and an RGB+D scanner as described above with respect to FIG. 1. For example, embodiments may employ any other imaging modalities (e.g., a magnetic resonance scanner, a positron-emission scanner, etc.) for acquiring surface data.

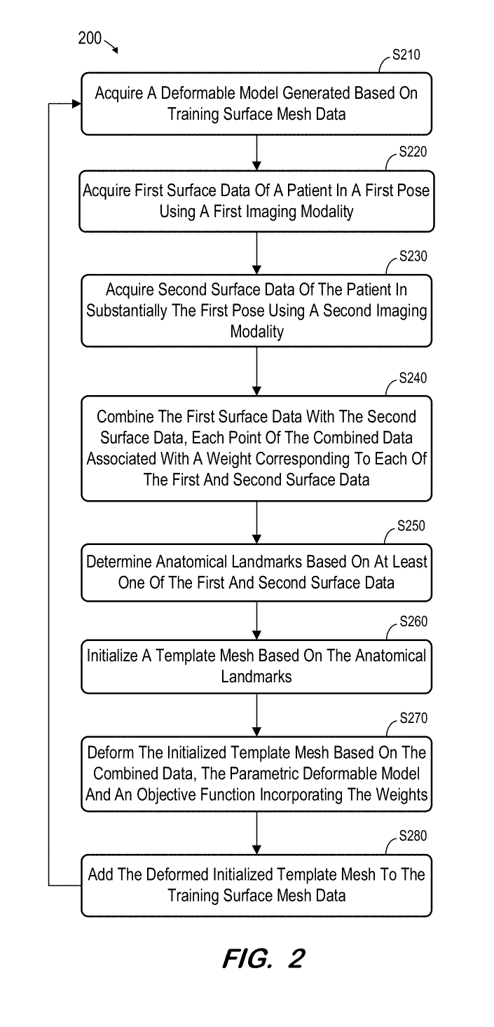

FIG. 2 is a flow diagram of process 200 according to some embodiments. Process 200 and the other processes described herein may be performed using any suitable combination of hardware, software or manual means. Software embodying these processes may be stored by any non-transitory tangible medium, including a fixed disk, a floppy disk, a CD, a DVD, a Flash drive, or a magnetic tape. Examples of these processes will be described below with respect to the elements of system 1, but embodiments are not limited thereto.

Initially, at S210, a deformable model of human body surface data is acquired. Embodiments are not limited to any particular type of deformable model. Embodiments are also not limited to any particular systems for training such a deformable model. Moreover, S210 may comprise generating the deformable model or simply acquiring the deformable model from another system.

According to some embodiments, the deformable model comprises a parametric deformable model (PDM) which divides human body deformation into separate pose and shape deformations, and where the pose deformation is further divided into rigid and non-rigid deformations. According to some embodiments, the PDM represents the human body using a polygon mesh. A polygon mesh is a collection of vertices and edges that defines the shape of an object. A mesh will be denoted herein as M^{X}=(P^{X}, V^{X}), where the P^{X}=(p₁, . . . , pₖ) represents the vertices and V^{X}=(v₁, . . . , vₖ) includes the vertex indices that composed of the edges of the current polygon.

Assuming the use of triangles as the polygons in the mesh, each triangle tₖ may be represented using the three vertices (p_{1,k}, p_{2,k}, p_{3,k}) and three edges (v_{1,k}, v_{2,k}, v_{3,k}). Triangles tₖⁱ in any given mesh Mⁱ can be represented as the triangles of the template mesh \hat{M} with some deformations. Denoting the triangles in the template mesh as \hat{t}ₖ and the two edges of each triangle as \hat{v}_{k,j}, j=2, 3, the triangles in Mⁱ may be represented as:

v_{k,j}ⁱ=R_{l[k]}ⁱSₖⁱQₖⁱ\hat{v}_{k,j} (1)

where R_{l[k]} is the rigid rotation matrix that exhibits the same value to all the triangles belonging to the same body part l. Sₖⁱ is the shape deformation matrix and Qₖⁱ is the pose deformation matrix. Embodiments are not limited to polygon meshes using triangles, as other polygons (e.g., tetrahedrons) may be also be used in a polygon mesh.

To learn the pose deformation model, a regression function is learned for each triangle tₖ, which estimates the pose deformation matrix Q as a function of the twists of its two nearest joints Δr_{l[k]}ⁱ,

$${Q}_{k , l [ m ]}^{i} = {Γ}_{{a}_{k , l [ m ]}}^{T} ( Δ {r}_{l [ k ]}^{i} ) = {a}_{k , l [ m ]}^{T} \cdot [ \begin{matrix}Δ {r}_{l [ k ]}^{i} \\ 1\end{matrix} ] \qquad ( 2 )$$

In the above equation, Δr can be calculated from the rigid rotation matrix R. If Q is given, the regression parameter a can be easily calculated. However, the non-rigid deformation matrix Q for each triangle is unknown. Accordingly, the deformation matrices for each of the triangles may be solved by solving an optimization problem that minimizes the distance between the deformed template mesh and the training mesh data with a smoothness constraint. This optimization problem may be expressed in some embodiments as:

$${argmin}_{\{ {Q}_{1}^{i} … {Q}_{P}^{i} \}} {\sum }_{k} {\sum }_{j = 2 , 3} {‖ {R}_{k}^{i} {Q}_{k}^{i} {\hat{v}}_{k , j} - {v}_{k , j}^{i} ‖}^{2} + {w}_{s} {\sum }_{{k}_{1} , {k}_{2} adj} I ( {l}_{{k}_{1}} = {l}_{{k}_{2}} ) \cdot {‖ {Q}_{{k}_{1}}^{i} - {Q}_{{k}_{2}}^{i} ‖}^{2} \qquad ( 3 )$$

where the first term minimizes the distance between the deformed template mesh and the training mesh data and the second term is a smoothness constraint that prefers similar deformations in adjacent triangles that belong to the same body part. wₛ is a weight that can be used to tune the smoothness constraint and I(lₖ_{₁}=lₖ_{₂}) is equal to the identity matrix I if the adjacent triangles belong to the same body part and equal to zero if the adjacent triangles do not belong to the same body part.

After training of the pose deformation model, the mesh model can be manipulated to form different body poses by initialing the rigid rotation matrix R with different values. In order to learn the shape deformation from a set of training data, principle component analysis (PCA) is employed to model shape deformation matrices as a linear combination of a small set of eigenspaces,

Sₖⁱ=ô_{U}_{ₖ}_{,μ}_{ₖ}(βₖⁱ)=Uₖβₖⁱ+μₖ (4)

Similar to the pose estimation, the shape deformation matrix S for each triangle is unknown. The matrix S may be estimated using an optimization problem that minimizes the distance between the deformed template mesh and the training mesh data subject to a smoothness constraint:

$${argmin}_{{S}^{i}} {\sum }_{k} {\sum }_{j = 2 , 3} {‖ {R}_{k}^{i} {S}_{k}^{i} {Q}_{k}^{i} {\hat{v}}_{k , j} - {v}_{k , j}^{i} ‖}^{2} + {w}_{s} {\sum }_{{k}_{1} , {k}_{2} adj} {‖ {S}_{{k}_{1}}^{i} - {S}_{{k}_{2}}^{i} ‖}^{2} \qquad ( 5 )$$

where the first term minimizes the distance between the deformed template mesh and the training mesh data, and the second term is a smoothness constraint that prefers similar shape deformations in adjacent triangles.

Once the PCA parameters (i.e., set of eigenvectors) are obtained, the mesh model can be manipulated to form different body shapes (tall to short, underweight to overweight, strong to slim, etc.) by perturbing β.

The process to train the pose deformation model and shape deformation model for the PDM requires many three-dimensional training meshes. The meshes may be generated from real human models, using a high-precision laser scanner to capture partial views of each person from different viewing angles. A registration algorithm is then applied to construct a full body surface model for each person from the partial views. Additional processing may be required to fill holes, remove noise, and smooth surfaces. Synthetic human models may also be generated via three-dimensional rendering software and used to train the PDM.

Returning to process 200, first surface data of the patient is acquired using a first imaging modality at S220 while the patient resides in a first pose. It will be assumed that, prior to S220, the patient is positioned for imaging according to known techniques. For example, and with reference to the elements of system 1, patient 15 is positioned on table 16 to place a particular volume of patient 15 in a particular relationship to scanner 20 and between radiation source 11 and radiation detector 13. System 30 may assist in adjusting table 16 to position the patient volume as desired. As is known in the art, such positioning may be based on a location of a volume of interest, on positioning markers located on patient 15, on a previously-acquired planning image, and/or on a portal image acquired after an initial positioning of patient 15 on table 16.

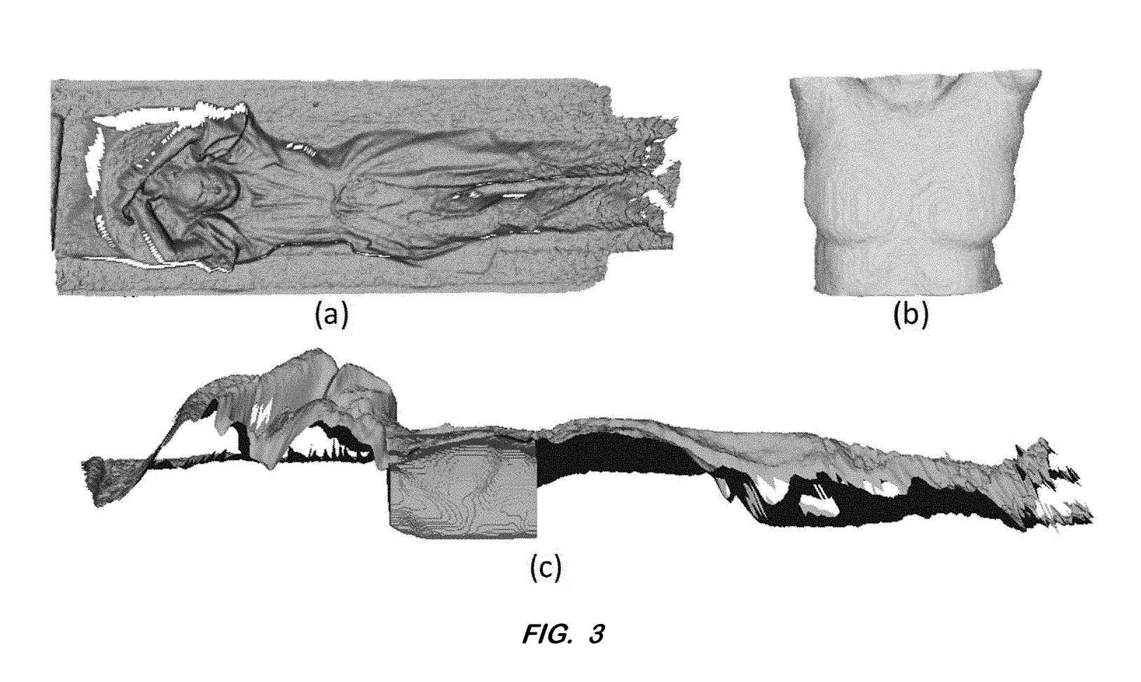

It will be assumed that scanner 20 is used to acquire the first surface data. The first surface data may therefore comprise RGB-D image data as described above. Portion (a) of FIG. 3 illustrates a top view of the first surface data acquired by scanner 20 according to some embodiments.

According to some embodiments, the RGB-D image data is converted to a 3D point cloud. In particular, the depth image of the RGB-D image data is used to map each pixel in the RGB image to a three-dimensional location, resulting in a 3D point cloud representing the patient. Each point in the point cloud may specify the three-dimensional coordinate of a surface point. The point cloud surface data may be denoted as P^{X}=(p₁, . . . , pₖ).

Second surface data of the patient is acquired at S230, while the patient resides substantially in the first pose. In some embodiments of S230, radiation source 11 is powered by a high-powered generator to emit x-ray radiation toward radiation detector 13 at various projection angles. The parameters of the x-ray radiation emission (e.g., timing, x-ray tube voltage, dosage) may be controlled by system control program 23 as is known in the art. Radiation detector 13 receives the emitted radiation and produces a set of data (i.e., a projection image) for each projection angle. The projection image may be received by system 30 and stored among CT frames 42 in either raw form or after any suitable pre-processing (e.g., denoising filters, median filters and low-pass filters).

The frames are reconstructed using known techniques to generate a three-dimensional CT image. Patient surface data is extracted from the three-dimensional CT image as is also known. Portion (b) of FIG. 3 illustrates patient skin surface reconstructed from the two-dimensional CT images according to some embodiments.

Next, at S240, the first surface data is combined with the second surface data. The combination may be based on registration of the two sets of data as is known in the art. Registration may be based on calibration data which represents a transform between the frame of reference of scanner 20 and a frame of reference of imaging system 10, and/or on detection of correspondences within the sets of surface data.

Portion (c) of FIG. 3 illustrates thusly-combined surface data according to some embodiments, and in which the patient is substantially in the same pose during acquisition of each set of surface data. As shown, the first surface data acquired by scanner 20 captures only upper surface information but over the full length of the patient, while the second (i.e., CT) surface data captures surface information over the full circumference of only a portion of the torso of the patient.

According to some embodiments, each point of the combined data is associated with a weight. A weight represents the degree to which a corresponding point value from each set of data should contribute to the value of the combined point. The weights of all the points, taken together, may comprise a “heatmap”. According to some embodiments, points located in the areas of the torso scanned by radiation source 11 and detector 13 may be weighted more heavily (or completely) toward the CT surface data while other points may be weighted more heavily (or completely) toward the RGB-D data.

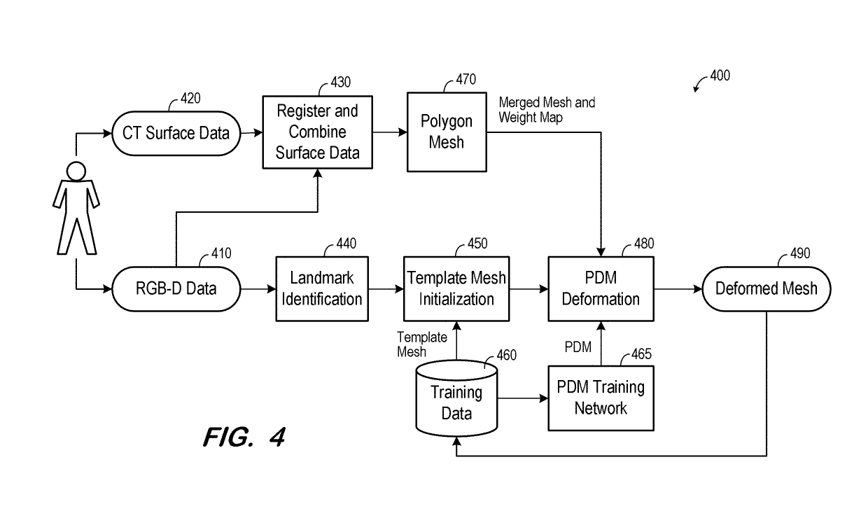

FIG. 4 illustrates system 400 implementing process 200 according to some embodiments. System 400 comprises software modules to perform the processes described herein. RGB-D data 410 may comprise the first surface data acquired at S220, while CT surface data 420 may comprise the second surface data acquired at S230. Module 430 is used to register and combine the two sets of surface data as described above.

Anatomical landmarks are determined based on at least one of the first and second surface data at S250. In the implementation of system 400, the anatomical landmarks are identified by module 440 based on RGB-D data 410. For example, module 440 may identify anatomical landmarks based on a point cloud generated from the RGB-D data.

According to some embodiments, joint landmarks are detected in the point cloud using machine learning-based classifiers trained based on annotated training data. For example, a respective probabilistic boosting tree (PBT) classifier can be trained for each of the joint landmarks and each joint landmark can be detected by scanning the point cloud using the respective trained PBT classifier. In some embodiments, the relative locations of the landmarks can be utilized in the landmark detection. For example, the trained classifiers for each of the joint landmarks can be connected in a discriminative anatomical network (DAN) to take into account the relative locations of the landmarks, or the trained classifiers can be applied to the point cloud in a predetermined order where each landmark that is detected helps to narrow the search range for the subsequent landmarks. In some embodiments, PBT classifiers can be trained to detect a plurality of body parts (e.g., head, torso, pelvis) and the detected body parts can be used to constrain the search range for the PBT classifiers which are used to detect the joint landmarks.

At S260, a template mesh model is initialized in the point cloud using the detected anatomical landmarks. With reference to system 400, a template mesh may be selected from the meshes in training data 460 and initialized by module 450. The selected mesh may exhibit a regular (e.g., average or median) body size and a neutral pose. Training data 460 may comprise data used to train the acquired PDM as described above. In this regard, FIG. 4 illustrates the generation of the PDM by PDM training network 465 based on training data 460. According to some embodiments, the PDM is acquired from a separate system, and/or the data used to train the PDM is difference from the data from which the template mesh is selected at S260.

According to some embodiments, each template mesh of training data 460 is divided into a plurality of body parts and a corresponding location for each of a plurality of joint landmarks on the template mesh is stored. The template mesh may be initialized in the point cloud at S260 by calculating a rigid transformation of the template mesh to the point cloud that minimizes error between the detected locations of the joint landmarks in the point cloud and the corresponding locations of the joint landmarks in the template mesh. This rigid transformation provides an initial rigid rotation matrix R, which when applied to the template mesh results in an initialized mesh.

Next, at S270, a new mesh is generated by deforming the template mesh based on the combined surface data, the PDM and an objective function incorporating the above-mentioned weights. Generally, the template mesh is deformed by module 480 to fit the combined surface data using the trained PDM.

As described above, the PDM is trained by training a pose deformation model and a shape deformation model from the training data. In contrast, the trained PDM is used to fine-tune the initialized mesh in S270. Given the combined (and partial) surface data and the respective weights, PDM deformation module 480 generates a full three-dimensional deformed mesh 490 by minimizing the objective function:

$${argmin}_{\{ β , Δ r , {y}_{k} \}} {\sum }_{k} {\sum }_{j = 2 , 3} {‖ {R}_{k} {\hat{O}}_{U , μ} ( β ) {Γ}_{{a}_{k}} ( Δ {r}_{l [ k ]} ) {\hat{v}}_{k , j} - ( {y}_{j , k} - {y}_{1 , k} ) ‖}^{2} + {w}_{z} {{\sum }_{l = 1}}^{L} {w}_{l} {‖ {y}_{l} - {z}_{l} ‖}^{2} \qquad ( 6 )$$

where Rₖ is the rigid rotation matrix, ô_{U}_{_{,μ}} (β) is the trained shape deformation model, Γₐ_{ₖ} (Δr_{l[k]}) is the trained pose deformation model, \hat{v}_{k,j} denotes edges of a triangle in the template mesh, y denotes vertices of the estimated avatar mesh model, and L is the set of correspondences between the avatar vertex yₗ and the corresponding point zₗ, in the 3D point cloud Z. The first term of th3 objective function defines the mesh output to be consistent with the learned PDM model and the second term regulates the optimization to find the set that best fits the input point cloud. To balance the importance of the two terms, a weighting term w_{Z} is applied.

A model generates a different value of wₗ for each registered point. wₗ represents how reliable the surface point l of each of the two datasets can be trusted. For example, the value of wₗ for the CT surface data than the value of wₗ for the RGB-D surface data in that region.

The above objective function includes three parameter sets (R, Y and β) to be optimized, thereby forming a standard non-linear and non-convex optimization problem. In order to avoid the possibility of converging to a sub-optimal solution, some embodiments utilize an iterative process to optimize the three parameters. In particular, the three sets of parameters are treated separately, optimizing only one of them at a time while keeping the other two fixed. According to some embodiments, a three-step optimization can be performed as follows:
Optimize R with S and Y fixed, then update ΔR and Q accordingly.
Optimize Y with R and S fixed.
Optimize S with R, Q and Y fixed.

In step (1) of three-step optimization procedure, the rigid rotation matrix R is optimized the objective function while the shape deformation S and the vertices Y of the estimated avatar mesh model are fixed. This results in an updated value of ΔR for each triangle in the estimated avatar mesh model and the pose deformation Q for each triangle is updated based on the updated ΔR using the trained posed deformation model Γₐ_{ₖ} (Δr_{l[k]}). Accordingly, step (1) of the optimization procedure optimizes the pose of the estimated avatar model.

In step (2) of the three-step optimization procedure, the locations of the vertices Y of the estimated avatar mesh are optimized using the objective function while the shape deformation S and rigid rotation matrix R (and pose deformation) are fixed. This step is a adjusts the locations of the vertices Y to better match the point cloud. In step (3) of the optimization procedure, the shape deformation S is optimized using the objective function while the rigid rotation matrix R, the pose deformation Q, and the vertices Y of the estimated avatar mesh are fixed. In particular, first principal component β is adjusted to find the shape deformation calculated using the trained deformation model ô_{U}_{_{,μ}} (β) that minimizes the objective function. Accordingly, the three-step optimization procedure first finds an optimal pose deformation, then performs fine-tuning adjustments of the vertices of the estimated avatar model, and then finds an optimal shape deformation. This three-step optimization procedure can be iterated a plurality of times. For example, the three-step optimization procedure can be iterated a predetermined number of times or can be iterated until it converges.

Evaluation of the second term in the objective function includes a determination of correspondences between the point cloud and the determined landmarks. An initial R is estimated based on the determined anatomical landmarks. Then, the above three-step optimization procedure is iterated a plurality of times to generate a current estimated mesh model, M_{curr}, where only the joint landmarks are used to find the correspondences. For example, the three-step optimization procedure using only the joint landmarks to find the correspondences can be iterated a predetermined number of times or can be iterated until it converges. Next, a registration algorithm based on, for example, the Iterative Closest Point algorithm can be performed to obtain a full registration between the point cloud and the current mesh model M_{curr}. Once the registration between the point cloud and the current mesh model M_{curr} is performed, correspondences between corresponding pairs of points in the point cloud and the current mesh model having a distance ∥yₗ−zₗ∥ larger than a predetermined threshold are removed. The remaining correspondences are then used to estimate a new rigid rotation matrix R, and the three-step optimization procedure is repeated. This optimization-registration process is iterated until convergence.

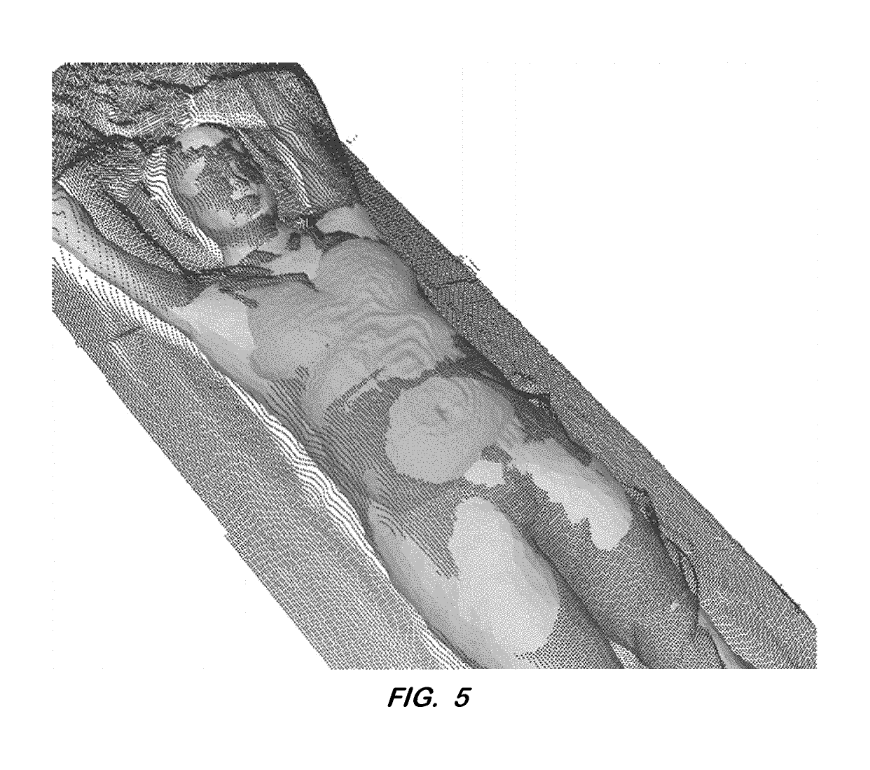

FIG. 5 illustrates an example of a deformable person mesh template aligned with RGB-D data (dense point cloud) and skin surface data obtained from a CT scan (shaded).

According to some embodiments, the deformed mesh is added to the training data at S280. Accordingly, the deformed mesh may be used as described above in subsequent training of the PDM. The deformed mesh may also or alternatively be used by a planning or positioning system to plan treatment or position a patient for treatment. As described above, the deformed mesh may provide improved estimation of internal patient anatomy with respect to conventional surface scanning.

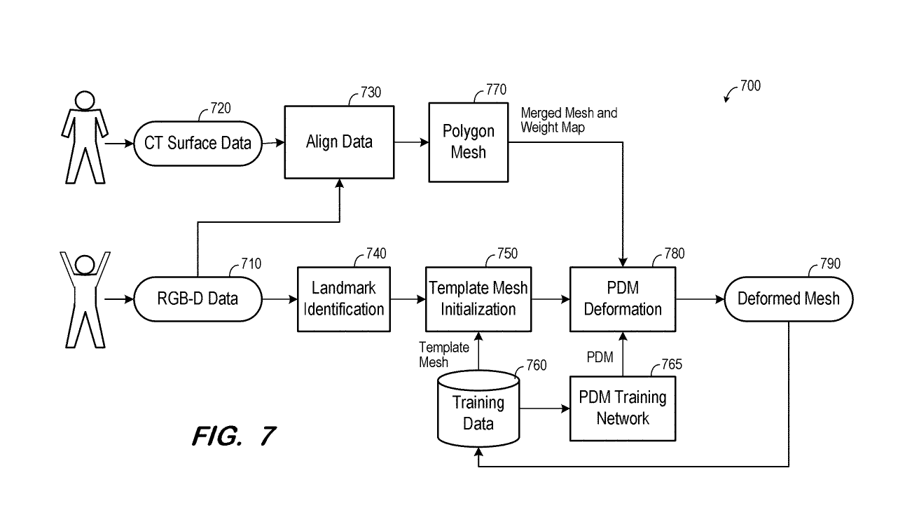

FIG. 7 illustrates system 700, which deforms a template mesh to simultaneously fit to data from different imaging modalities without assuming the data to be previously aligned or obtained with the patient in the same pose. More particularly, system 700 differs from system 400 (and process 200) in that CT surface data 720 represents the patient disposed in a first pose and RGB-D data 710 represents the (same) patient in a different, second, pose. Moreover, system 700 does not assume the existence of calibration between the system which acquires RGB-D data 710 and the system which acquires CT surface data 720. Accordingly, module 730 merely attempts to align data 710 and data 720 using known image alignment techniques.

To address the variation in pose across the different data acquisitions, the objective function implemented by PDM deformation module 780 is modified to incorporate for pose variation, which is modeled using R and Q matrices. Shape parameters, modeled using the S matrix, are considered to be the same across each data acquisition, based on the underlying assumption that the data acquisitions are different observations of the same physical person. Accordingly, the shape (body regions and size) should be the same.

The merged mesh may include two sets of limbs due to the different poses. Some embodiments therefore include a determination of which set of limbs to trust and to assign weights accordingly.

Those in the art will appreciate that various adaptations and modifications of the above-described embodiments can be configured without departing from the scope and spirit of the claims. Therefore, it is to be understood that the claims may be practiced other than as specifically described herein.

What is claimed is:

1. A method comprising:
acquiring first surface data of a patient in a first pose using a first imaging modality;
acquiring second surface data of the patient in a second pose using a second imaging modality;
combining the first surface data and the second surface data to generate combined surface data;
for each point of the combined surface data, determining a weight associated with the first surface data and a weight associated with the second surface data;
detecting a plurality of anatomical landmarks based on the first surface data;
initializing a first polygon mesh by aligning a template polygon mesh to the combined surface data based on the detected anatomical landmarks;
deforming the first polygon mesh based on the combined surface data, a trained parametric deformable model, and the determined weights; and
storing the deformed first polygon mesh.

2. A method according to claim 1, further comprising:
re-positioning the patient based on the deformed first polygon mesh.

3. A method according to claim 1, further comprising:
re-training the parametric deformable model based on the deformed first polygon mesh.

4. A method according to claim 1, wherein the first surface data comprises a red, green, blue (RGB) image and a depth image, the method further comprising:
for each of a plurality of pixels in the RGB image, mapping the pixel to a location in a point cloud based on a corresponding depth value in the depth image,
wherein detecting the plurality of anatomical landmarks based on the first surface data comprises detecting the plurality of anatomical landmarks based on the point cloud.

5. A method according to claim 1, wherein the first pose and the second pose are substantially identical, and wherein deforming the first polygon mesh based on the combined surface data, a trained parametric deformable model, and the determined weights comprises:
deforming the first polygon mesh based on the combined surface data, a trained parametric deformable model, and the objective function:
$${argmin}_{\{ β , Δ r , {y}_{k} \}} {\sum }_{k} {\sum }_{j = 2 , 3} {‖ {R}_{k} {\hat{O}}_{U , μ} ( β ) {Γ}_{{a}_{k}} ( Δ {r}_{l [ k ]} ) {\hat{v}}_{k , j} - ( {y}_{j , k} - {y}_{1 , k} ) ‖}^{2} + {w}_{z} {{\sum }_{l = 1}}^{L} {w}_{l} {‖ {y}_{l} - {z}_{l} ‖}^{2} .$$

6. A method according to claim 1, wherein deforming the first polygon mesh based on the combined surface data, a trained parametric deformable model, and the determined weights comprises:
deforming the first polygon mesh based on the combined surface data, a trained parametric deformable model, and the objective function:
$${argmin}_{\{ β , Δ r , {y}_{k} \}} {\sum }_{k} {\sum }_{j = 2 , 3} {‖ {R}_{k} {\hat{O}}_{U , μ} ( β ) {Γ}_{{a}_{k}} ( Δ {r}_{l [ k ]} ) {\hat{v}}_{k , j} - ( {y}_{j , k} - {y}_{1 , k} ) ‖}^{2} + {w}_{z} {{\sum }_{l = 1}}^{L} {w}_{l} {‖ {y}_{l} - {z}_{l} ‖}^{2} .$$

7. A method according to claim 1, further comprising:
determining a patient isocenter based on the deformed first polygon mesh; and
determining an imaging plan based on the patient isocenter.

8. A system comprising:
a first image acquisition system to acquire first surface data of a patient in a first pose using a first imaging modality;
a second image acquisition system to acquire second surface data of the patient in a second pose using a second imaging modality;
a processor to:
combine the first surface data and the second surface data to generate combined surface data;
for each point of the combined surface data, determine a weight associated with the first surface data and a weight associated with the second surface data;
detect a plurality of anatomical landmarks based on the first surface data;
initialize a first polygon mesh by aligning a template polygon mesh to the combined surface data based on the detected anatomical landmarks; and
deform the first polygon mesh based on the combined surface data, a trained parametric deformable model, and the determined weights; and
a storage device to store the deformed first polygon mesh.

9. A system to claim 8, the processor to further operate the system to re-position the patient based on the deformed first polygon mesh.

10. A system according to claim 8, the processor further to:
re-train the parametric deformable model based on the deformed first polygon mesh.

11. A system according to claim 8, wherein the first surface data comprises a red, green, blue (RGB) image and a depth image, the processor further to:
for each of a plurality of pixels in the RGB image, map the pixel to a location in a point cloud based on a corresponding depth value in the depth image,
wherein detection of the plurality of anatomical landmarks based on the first surface data comprises detection of the plurality of anatomical landmarks based on the point cloud.

12. A system according to claim 8, wherein the first pose and the second pose are substantially identical, and wherein deforming of the first polygon mesh based on the combined surface data, a trained parametric deformable model, and the determined weights comprises:
deforming of the first polygon mesh based on the combined surface data, a trained parametric deformable model, and the objective function:
$${argmin}_{\{ β , Δ r , {y}_{k} \}} {\sum }_{k} {\sum }_{j = 2 , 3} {‖ {R}_{k} {\hat{O}}_{U , μ} ( β ) {Γ}_{{a}_{k}} ( Δ {r}_{l [ k ]} ) {\hat{v}}_{k , j} - ( {y}_{j , k} - {y}_{1 , k} ) ‖}^{2} + {w}_{z} {{\sum }_{l = 1}}^{L} {w}_{l} {‖ {y}_{l} - {z}_{l} ‖}^{2} .$$

13. A system according to claim 8, wherein deforming of the first polygon mesh based on the combined surface data, a trained parametric deformable model, and the determined weights comprises:
deforming of the first polygon mesh based on the combined surface data, a trained parametric deformable model, and the objective function:
$${argmin}_{\{ β , Δ r , {y}_{k} \}} {\sum }_{k} {\sum }_{j = 2 , 3} {‖ {R}_{k} {\hat{O}}_{U , μ} ( β ) {Γ}_{{a}_{k}} ( Δ {r}_{l [ k ]} ) {\hat{v}}_{k , j} - ( {y}_{j , k} - {y}_{1 , k} ) ‖}^{2} + {w}_{z} {{\sum }_{l = 1}}^{L} {w}_{l} {‖ {y}_{l} - {z}_{l} ‖}^{2} .$$

14. A system according to claim 8, the processor further to:
determine a patient isocenter based on the deformed first polygon mesh; and
determine an imaging plan based on the patient isocenter.

15. A non-transitory computer-readable medium storing processor-executable process steps, the process steps executable by a processor to cause a system to:
acquire first surface data of a patient in a first pose using a first imaging modality;
acquire second surface data of the patient in a second pose using a second imaging modality;
combine the first surface data and the second surface data to generate combined surface data;
for each point of the combined surface data, determine a weight associated with the first surface data and a weight associated with the second surface data;
detect a plurality of anatomical landmarks based on the first surface data;
initialize a first polygon mesh by aligning a template polygon mesh to the combined surface data based on the detected anatomical landmarks;
deform the first polygon mesh based on the combined surface data, a trained parametric deformable model, and the determined weights; and
store the deformed first polygon mesh.

16. A medium according to claim 15, the processor further to:
re-train the parametric deformable model based on the deformed first polygon mesh.

17. A medium according to claim 15, wherein the first surface data comprises a red, green, blue (RGB) image and a depth image, the processor further to:
for each of a plurality of pixels in the RGB image, map the pixel to a location in a point cloud based on a corresponding depth value in the depth image,
wherein detection of the plurality of anatomical landmarks based on the first surface data comprises detection of the plurality of anatomical landmarks based on the point cloud.

18. A medium according to claim 15, wherein the first pose and the second pose are substantially identical, and wherein deforming of the first polygon mesh based on the combined surface data, a trained parametric deformable model, and the determined weights comprises:
deforming of the first polygon mesh based on the combined surface data, a trained parametric deformable model, and the objective function:
$${argmin}_{\{ β , Δ r , {y}_{k} \}} {\sum }_{k} {\sum }_{j = 2 , 3} {‖ {R}_{k} {\hat{O}}_{U , μ} ( β ) {Γ}_{{a}_{k}} ( Δ {r}_{l [ k ]} ) {\hat{v}}_{k , j} - ( {y}_{j , k} - {y}_{1 , k} ) ‖}^{2} + {w}_{z} {{\sum }_{l = 1}}^{L} {w}_{l} {‖ {y}_{l} - {z}_{l} ‖}^{2} .$$

19. A medium according to claim 15, wherein deforming of the first polygon mesh based on the combined surface data, a trained parametric deformable model, and the determined weights comprises:
deforming of the first polygon mesh based on the combined surface data, a trained parametric deformable model, and the objective function:
$${argmin}_{\{ β , Δ r , {y}_{k} \}} {\sum }_{k} {\sum }_{j = 2 , 3} {‖ {R}_{k} {\hat{O}}_{U , μ} ( β ) {Γ}_{{a}_{k}} ( Δ {r}_{l [ k ]} ) {\hat{v}}_{k , j} - ( {y}_{j , k} - {y}_{1 , k} ) ‖}^{2} + {w}_{z} {{\sum }_{l = 1}}^{L} {w}_{l} {‖ {y}_{l} - {z}_{l} ‖}^{2} .$$

20. A medium according to claim 15, the processor further to:
determine a patient isocenter based on the deformed first polygon mesh; and
determine an imaging plan based on the patient isocenter.
